(19) United States
(12) Patent Application Publication
(10) Pub. No.: US 2007/0212414 A1
(43) Pub. Date: Sep. 13, 2007

(54) Ethanol-resistant sustained release formulations

(75) Inventors: Anand R. Baichwal, Wappingers Falls, NY (US); Steve Labudzinski, Poughkeepsie, NY (US)

Correspondence Address: WILMER CUTLER PICKERING HALE AND DORR LLP, 60 STATE STREET, BOSTON, MA, 02109, US

(73) Assignee: Penwest Pharmaceuticals Co., Patterson, NY (US)

(21) Appl. No.: 11/370,699

(22) Filed: Mar. 8, 2006

(51) Int. Cl. A61K 9/22; A61K 38/22; A61K 31/485

(52) U.S. Cl. 424/468; 514/12; 514/282

(57) ABSTRACT

The invention provides formulations that resist dose dumping in the presence of ethanol and methods of use thereof. The formulations can be used to prevent dose dumping, to increase safety of drugs, and to reduce abuse of drugs prone to such abuse. The formulations comprise at least one drug and a sustained release delivery system. In one embodiment, the drug is an opioid.

### TECHNICAL FIELD

The invention provides sustained release formulations that maintain their dissolution properties when ingested or used concurrently with ethanol and methods of use thereof. The ethanol-resistant formulations comprise at least one drug and a sustained release delivery system.

### BACKGROUND

Sustained release drug formulations often contain higher amounts of a drug than conventional non-sustained release formulations. Functionality and safety of a sustained release formulation are based on a known controlled rate of drug release from the formulation over an extended period of time after administration, such as 8-24 hours. The drug release profile of a formulation often depends on the chemical environment of the sustained release formulation, for example, on pH, ionic strength and presence of solvents such as ethanol.

The relatively high amount of drug that is present in a sustained release formulation can, in some instances, harm a patient if the formulation releases the drug at a rate that is faster or slower than the intended controlled rate.

In most cases, failure of a sustained release formulation results in a rapid release of the drug. This rapid release is generally faster than the intended sustained release of the drug from the formulation, and is sometimes referred to as “dose dumping.”

Dose dumping can create severe consequences for a patient, including permanent harm and even death. Examples of drugs that can be fatal if the therapeutically beneficial dose is exceeded, e.g., by dose dumping, include pain medications such as opioids.

Oral dosage formulations are often taken with a commonly available beverage, such as water, juice, a carbonated beverage or an ethanol-containing beverage. An ethanol-containing beverage is commonly referred to as an alcoholic beverage, liquor, or simply alcohol. Examples of common alcoholic beverages include beer, wine, and hard liquors such as vodka, rum, or whiskey. Dose dumping in the presence of ethanol creates a safety concern because of the likelihood that a patient will ingest the formulation with an alcoholic beverage. An additional safety concern is that a patient will consume alcoholic beverages while being treated with the drug in the formulation, even if the patient does not ingest the formulation with an alcoholic beverage.

Patients who desire to abuse a drug, for example a drug that causes a euphoric effect, may want to intentionally induce dose dumping in order to magnify the euphoric effect of the drug. Furthermore, a person wanting to abuse a drug might already be abusing alcohol, which increases the likelihood of the sustained release formulation of the drug to be ingested or taken concurrently with an alcoholic beverage.

In 2005, several drugs were either withdrawn from the market or had their warning labels enhanced because of the effects of ethanol on the sustained release formulations of the drug.

For instance, the United States Food and Drug Administration (FDA) asked Purdue Pharma to withdraw Palladone® (hydromorphone hydrochloride) extended release capsules from the market because a study showed that when Palladone is taken with alcohol, its extended release formulation is damaged and can dose dump (cf. FDA Press Release of Jul. 13, 2005). FDA further warned that taking Palladone® with a single alcoholic drink could have fatal consequences for the patient.

Alpharma issued a press release reporting that FDA had requested it to expand warning information regarding alcohol in the labeling for KADIAN® (morphine sulfate extended release capsules). (cf. http://www.alpharma.com/pages/getpage.aspx?id=19D731C5-5017-4DF9-9A67-4F514C00B9DF) The enhanced warning was a result of in vitro studies showing that the extended release characteristics of KADIAN® are compromised in the presence of alcohol.

AVINZA® (morphine sulfate extended-release capsules) was found to have an increased risk of dose dumping when taken with ethanol. In vitro studies performed by the FDA showed that when AVINZA 30 mg was mixed with 900 mL of buffer solutions containing ethanol (20% and 40%), the dose of morphine that was released was alcohol concentration-dependent, leading to a more rapid release of morphine, which in vivo could result in the absorption of a potentially fatal dose of morphine. As a result, Ligand Pharmaceuticals Inc. revised several sections of the AVINZA® prescribing information to highlight and strengthen the warning that patients should not consume alcohol while taking AVINZA®. Additionally, patients were warned not to use prescription or non-prescription medications containing alcohol while on AVINZA® therapy. (c.f. http://www.fda.gov/medwatch/SAFETY/2005/safety05.htm#Avinza)

FDA has also indicated that for future sustained release products, in vitro testing for alcohol-induced undermining of sustained release characteristics may be advisable as a routine characterization test. Furthermore, FDA's position is that for certain drugs (e.g., drugs with a narrow therapeutic index or dire consequences of high Cₘₐₓ or low Cₘᵢₙ), alcohol sensitive sustained release formulations should not be approved. FDA prefers that formulations be made ethanol-resistant by design, rather than simply a confirmation that dose dumping does not occur through an in vivo study. (cf. Summary of FDA's position on alcohol-induced dose dumping as presented at the Pharmaceutical Sciences Advisory Committee Meeting Oct. 26, 2005)

According to the FDA, an in vivo alcohol resistance test is not the preferred approach due to potential harm the test could pose to a human subject. The preferred approach, according to the FDA, is an in vitro dissolution test in the presence of 40% ethanol. FDA is proposing classifying formulations into three groups: rugged, vulnerable and uncertain. At the Pharmaceutical Sciences Advisory Committee Meeting of Oct. 26, 2005, OPS (Office of Pharmaceutical Science) at the CDER (Center for Drug Evaluation and Research) personnel presented data showing that in a vulnerable formulation, a higher concentration of ethanol (e.g., 40%) is likely to trigger faster drug release than a lower concentration of ethanol (e.g., 20% or 4%). In FDA's example of a rugged formulation, the drug release from a formulation dissolved in 40% ethanol is actually slightly slower (although similar) compared to a control formulation dissolved in a medium without ethanol. (Presentations at the Pharmaceutical Sciences Advisory Committee Meeting Oct. 26, 2005)

Changes to product labeling (i.e., added warnings of the danger of taking the drug with alcohol) have only a limited effect and are not likely to deter a patient who intends to abuse the drug.

There is a need in the art for pharmaceutical formulations that resist ethanol-induced dose dumping. The invention is directed to this, as well as other, important ends.

### SUMMARY OF THE INVENTION

The invention provides ethanol-resistant pharmaceutical formulations and methods for increasing drug safety and reducing the potential for drug abuse. This can be achieved by providing, prescribing and/or administering to patients an effective amount of an ethanol-resistant drug formulation. The ethanol-resistant drug formulations are safer and have less potential for abuse when compared to commercially available formulations because their sustained release dissolution profile in an aqueous solution or in an ethanol-containing solution is essentially the same. In one embodiment, the drug in the ethanol-resistant formulation comprises an opioid compound or a derivative thereof.

The invention also provides ethanol-resistant pharmaceutical formulations and methods for preventing dose dumping. This can be achieved by providing, prescribing and/or administering to patients an effective amount of an ethanol-resistant drug formulation. The ethanol-resistant pharmaceutical formulations described herein do not dose dump in the presence of ethanol. In one embodiment, the drug in the ethanol-resistant formulation comprises an opioid compound or a derivative thereof.

In one aspect, the invention provides a method of preventing dose-dumping of a drug in the presence of ethanol comprising providing a patient, who may to consume ethanol while being treated with the drug, an effective amount of the drug in the form of an ethanol-resistant sustained release formulation comprising: the drug; and a sustained release delivery system, the delivery system comprising at least one heteropolysaccharide gum, at least one homopolysaccharide gum, and at least one pharmaceutical diluent, wherein the ethanol-resistant sustained release formulation essentially retains its sustained release dissolution profile in the presence of ethanol.

In another aspect, the invention provides a method of preventing dose-dumping of a drug in the presence of ethanol comprising providing a patient, who may to consume ethanol while being treated with the drug, an effective amount of the drug in the form of an ethanol-resistant sustained release formulation comprising: the drug; and a sustained release delivery system, the delivery system comprising at least one heteropolysaccharide gum, at least one cationic cross-linking compound selected from monovalent metal cations, multivalent metal cations and salts, and at least one pharmaceutical diluent, wherein the ethanol-resistant sustained release formulation essentially retains its sustained release dissolution profile in the presence of ethanol.

In one aspect, the invention provides a method of improving safety of a drug compared to conventional sustained release formulations in the presence of ethanol comprising providing a patient, who may to consume ethanol while being treated with the drug, an effective amount of the drug in the form of an improved-safety ethanol-resistant sustained release formulation comprising the drug; and a sustained release delivery system, the sustained release delivery system comprising at least one heteropolysaccharide gum, at least one homopolysaccharide gum and at least one pharmaceutical diluent, wherein the improved-safety is a result of ethanol-resistant sustained release properties of the formulation.

In another aspect, the invention provides a method of improving safety of a drug compared to conventional sustained release formulations in the presence of ethanol comprising providing a patient, who may to consume ethanol while being treated with the drug, an effective amount of the drug in the form of an improved-safety ethanol-resistant sustained release formulation comprising: the drug; and a sustained release delivery system, the delivery system comprising at least one heteropolysaccharide gum, at least one cationic cross-linking compound selected from monovalent metal cations, multivalent metal cations and salts, and at least one pharmaceutical diluent, wherein the improved-safety is a result of ethanol-resistant sustained release properties of the formulation.

In certain embodiments, the patient has a history of substance abuse, such as drug and/or alcohol abuse.

In other embodiments, the ethanol-resistant sustained release formulation is a solid dosage formulation, for example a tablet.

In some embodiments, the drug is an anti-depressant, a drug used to treat bipolar disorder, panic disorder, epilepsy, migraine, attention deficit hyperactivity disorder, and/or pain. In one embodiment, the drug is an opioid or a derivative thereof.

In one aspect, the invention provides a method for making a solid dosage ethanol-resistant sustained release formulation comprising: at least one drug; and a sustained release delivery system, wherein the sustained release delivery system comprises at least one heteropolysaccharide gum, at least one homopolysaccharide, and at least one pharmaceutical diluent, the method comprising: mixing the at least one heteropolysaccharide gum, the at least one homopolysaccharide gum and the at least one pharmaceutical diluent to form granules; mixing the granules with at least one drug or a pharmaceutically acceptable salt thereof to form a granulated composition; applying pressure to the granulated composition to make the formulation; and recording a dissolution profile of the ethanol-resistant formulation in an ethanol-containing solution.

In another aspect, the invention provides a method for making a solid dosage ethanol-resistant formulation comprising: at least one drug; and a sustained release delivery system, wherein the sustained release delivery system comprises at least one heteropolysaccharide gum, at least one cationic cross-linking compound selected from monovalent metal cations, multivalent metal cations and salts, and at least one pharmaceutical diluent, the method comprising: mixing the at least one heteropolysaccharide gum, the at least one cationic cross-linking compound selected from monovalent metal cations, multivalent metal cations and salts, and the at least one pharmaceutical diluent to form granules; mixing the granules with at least one drug or a pharmaceutically acceptable salt thereof to form a granulated composition; applying pressure to the granulated composition to make the formulation; and recording a dissolution profile of the ethanol-resistant formulation in an ethanol-containing solution.

In one aspect, the invention provides a method for treating a patient comprising providing a patient having a history of substance abuse an effective amount of the drug in the form of an ethanol-resistant sustained release formulation comprising: at least one drug; and a sustained release delivery system, the delivery system comprising at least one heteropolysaccharide gum, at least one homopolysaccharide gum and at least one pharmaceutical diluent, wherein the ethanol-resistant sustained release formulation essentially retains its sustained release dissolution profile in the presence of ethanol.

In another aspect, the invention provides a method for treating a patient comprising providing a patient having a history of substance abuse an effective amount of the drug in the form of an ethanol-resistant sustained release formulation comprising: at least one drug; and a sustained release delivery system, the delivery system comprising at least one heteropolysaccharide gum, at least one cationic cross-linking compound selected from monovalent metal cations, multivalent metal cations and salts, and at least one pharmaceutical diluent, wherein the ethanol-resistant sustained release formulation essentially retains its sustained release dissolution profile in the presence of ethanol.

These and other aspects of the invention are described in detail herein.

### BRIEF DESCRIPTION OF THE DRAWINGS

FIG. 1 shows dissolution profiles of different lots of TIMERx-N® with a reference drug in a buffer.

FIG. 2 shows dissolution profiles of different lots of TIMERx-N® with a reference drug in a buffer and in a 40% ethanol/0.1N HCl solution.

### DETAILED DESCRIPTION

Definitions

As used herein, the term “dose dumping” refers to a rapid release of a drug from a sustained release formulation. This rapid release is generally faster than a sustained release of a drug from the formulation. Dose dumping also refers to a release having a peak concentration of the drug higher than the peak concentration of the intended sustained release of the drug.

As used herein, the term “sustained release” means that the drug is released from the formulation at a controlled rate so that therapeutically beneficial blood levels (but below toxic levels) of the drug are maintained over an extended period of time.

As used herein, terms “sustained release”, “extended release” and “controlled release” are meant to be synonyms, i.e., have identical meaning.

As used herein, the term “liquids” includes, for example, gastrointestinal fluids, aqueous solutions (such as those used for in vitro dissolution testing), and mucosas (e.g., of the mouth, nose, lungs, esophagus, and the like).

As used herein, the term “ethanol-resistant formulation” refers to a formulation that has its sustained release properties substantially unmodified in the presence of ethanol.

As used herein, the terms “substantially unmodified” and “essentially retains” refer to a parameter value or a series of parameter values being in the range of about 80% to about 125% of the previous or original parameter value or the series of previous or original parameter values.

As used herein, the term “ethanol-resistant” refers to a property of a formulation that is substantially unmodified in the presence of ethanol.

As used herein, the term “abuse-potential drug” includes pharmaceutically active substances having the capacity to produce the kind of physical dependence in which drug withdrawal causes sufficient distress to bring about drug-seeking behavior; the ability to suppress withdrawal symptoms caused by withdrawal from other agents; the degree to which it induces euphoria (e.g., similar to that produced by morphine and other opioids); the patterns of toxicity that occur when the drug is dosed above its normal therapeutic range; and physical characteristics of the drugs, such as water solubility. The physical characteristics of the drug may determine whether the drug is likely to be abused by inhalation or parenteral routes. An abuse-potential drug includes stereoisomers thereof, metabolites thereof, salts thereof, ethers thereof, esters thereof and/or derivatives thereof (pharmaceutically acceptable salts thereof). An opioid is an embodiment of an abuse-potential drug. Other narcotics are apparent to those of ordinary skill in the art and are understood to fall within the scope of “abuse-potential drug.”

As used herein, the term “opioid” includes stereoisomers thereof, metabolites thereof, salts thereof, ethers thereof, esters thereof and/or derivatives thereof (e.g., pharmaceutically acceptable salts thereof). The opioids may be mu-antagonists and/or mixed mu-agonists/antagonists. Exemplary opioids include alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, stereoisomers thereof, metabolites thereof, salts thereof, ethers thereof, esters thereof, and/or derivatives thereof. In some embodiments, the opioid is morphine, codeine, hydromorphone, hydrocodone, oxycodone, dihydrocodeine, dihydromorphine, oxymorphone, 6-hydroxyoxymorphone (including 6-α-hydroxyoxymorphone and/or 6-β-hydroxyoxymorphone), or tramadol.

As used herein, the term “condition” includes any disease or a collection of symptoms that requires treatment with a drug. Exemplary conditions include panic disorder (with or without agoraphobia), bipolar disorder (manic depressive illness), acute manic or mixed episodes associated with bipolar disorder, epilepsy, migraine, attention deficit hyperactivity disorder (ADHD), depression and painAs used herein, the term “drug” includes any chemical or biological compound used for alleviating symptoms, treating or preventing a condition. Drugs suited for the ethanol-resistant formulations described herein include, but are not limited to, alprazolam (XANAX XR®), lithium carbonate (LITHOBID®), divalproex sodium (DEPAKOTE®), neutral sulfate salts of dextroamphetamine and amphetamine, with the dextro isomer of amphetamine saccharate and d,l-amphetamine aspartate monohydrate (ADDERALL XR®), tramadol hydrochloride (TRAMADOL ER®) and opioids.

The drug may be in the form of any pharmaceutically acceptable salt known in the art. Exemplary pharmaceutically acceptable salts include hydrochloric, sulfuric, nitric, phosphoric, hydrobromic, maleric, malic, ascorbic, citric, tartaric, pamoic, lauric, stearic, palmitic, oleic, myristic, lauryl sulfuric, napthalinesulfonic, linoleic, linolenic acid, and the like.

The ethanol-resistant sustained release formulations of drugs are administered in an amount sufficient to alleviate symptoms, treat or prevent a condition for an extended period of time, for example about 8 hours to about 24 hours, or for a period of about 12 hours to about 24 hours. The sustained release oral solid dosage formulations described herein may be administered one to four times a day, once or twice daily, or only once daily.

The ethanol-resistant sustained release formulations of opioids are administered in an amount sufficient to alleviate pain for an extended period of time, for example about 8 hours to about 24 hours, or for a period of about 12 hours to about 24 hours. The opioid sustained release oral solid dosage formulations described herein may be administered one to four times a day, once or twice daily, or only once daily.

The pain may be minor to moderate to severe, or moderate to severe. The pain may be acute or chronic. The pain may be associated with, for example, cancer, autoimmune diseases, infections, surgical traumas, or accidental traumas. The patient may be an animal, a mammal, or a human.

An effective amount of a drug is an amount sufficient to eliminate or to alleviate symptoms of a condition (e.g., reduce the pain compared to the pain present prior to administration of the opioid sustained release formulation).

Ethanol-Resistant Properties of the Sustained Release Formulations

It has been discovered that the sustained release formulations described herein retain their sustained release dissolution properties in the presence of ethanol.

Without intending to be bound by any theory, the physicochemical properties of the hydrophilic compound (e.g., xanthan gum) cross-linked by a cross-linking agent (e.g., locust bean gum), are such that they together form a gum or gum-like matrix, which is insoluble or substantially insoluble in ethanol. These solubility properties of the formulation may be attributed to the hydrophilic nature of the sustained release delivery system, which in one embodiment comprises one or more heteropolysaccharide gums and one or more homopolysaccharide gums, and in another embodiment comprises or one or more heteropolysaccharide gums, and one or more monovalent cations, multivalent cations, and/or salts. Small amounts of hydrophobic agents (e.g., hydrophobic polymers such as ethylcellulose), do not substantially modify the dissolution properties of the formulation in ethanol, presumably because the sustained release delivery system retains its hydrophilic character. Properties of the drug are not likely to affect the gum or gum-like properties of the matrix, making the formulations described herein suitable and/or adaptable to a wide range of drugs.

Several factors are believed to affect the release of a drug from the formulation in the presence of ethanol: solubility of the drug in ethanol, materials comprising the formulation (e.g., hydrophilic compounds are more resistant to ethanol than hydrophobic compounds), and dosage form of the formulation (e.g., tablets are more resistant to ethanol than capsules).

Additional factors believed to affect the release of a drug from the formulation in the presence of ethanol are: degree of compression of the dosage (e.g., harder tablets are more resistant to ethanol than softer tablets), tablet composition (e.g., monolithic tablet compositions are less resistant to ethanol than multiparticulate particle unit dosage forms enclosed in a gelatin capsule), and presence of a gel-like coating which is resistant to dissolution in ethanol (e.g., certain celluloses).

The sustained release formulations described herein can, therefore, be used to prevent or substantially reduce any undesired effects of ethanol on the release of the drug from a formulation. Exemplary undesired effects include dose dumping and altered sustained release dissolution profiles.

Alteration of a sustained release profile can be exhibited, for example, by a high peak drug concentration (Cₘₐₓ), which can increase the safety risk of a drug, and/or a low drug concentration at the end of the therapeutic period (Cₘᵢₙ), which can reduce the efficacy of the drug.

In an exemplary scenario, a formulation with an altered sustained release profile by ethanol may, for example, release a larger amount of the drug shortly after administration (e.g., within 0-6 hours), resulting in a higher-than-intended Cₘₐₓ. If the drug is toxic, a higher-than-intended Cₘₐₓ can lead to harmful side effects for the patient, including death. As a consequence of this rapid release, less drug is available for subsequent release, resulting in a lower-than-intended Cₘᵢₙ at the end of the therapeutic period (i.e., just prior to administration of a subsequent dose). A lower-than-intended Cₘᵢₙ can result in reduced efficacy or even inefficacy of the drug, which can result in recurrence of a condition in a patient.

A higher-than-intended peak drug concentration Cₘₐₓ can be, for example, a concentration more than 50% higher than intended Cₘₐₓ. A lower-than-intended Cₘᵢₙ concentration can be, for example, a concentration more than 50% lower than intended Cₘᵢₙ.

At the Pharmaceutical Sciences Advisory Committee Meeting of Oct. 26, 2005, FDA personnel presented results of a post-approval in vivo study of a known drug. The study showed that taking the drug with a beverage containing 40% alcohol led to a five-fold increase in Cₘₐₓ and taking the same drug with a beverage containing 20% alcohol led to a doubling of Cₘₐₓ. Taking the drug with a beverage containing 5% alcohol led to a small mean effect, but at least one subject doubled their Cₘₐₓ (http://www.fda.gov/ohrms/dockets/ac/05/slides/2005-4187S2_{—}02_Hussain_files/frame.htm).

The sustained release formulations described herein can, therefore, be used to increase safety of drugs with potentially harmful effects at high concentrations and to reduce abuse of drugs producing a euphoric effect, such as opioids. The formulations described herein can also be used to reduce or prevent harm to a patient in situations where a reduced level of a drug (e.g., lower than the therapeutically beneficial level) can adversely affect the health of the patient. The formulations described herein can be useful for formulation of narrow therapeutic range drugs, sometimes referred to as narrow therapeutic index drugs.

If a formulation described herein is ingested with an alcoholic beverage, or ingested by a patient prior to or after consumption of an alcoholic beverage, the formulation will essentially retain its sustained release properties and will slowly release the drug from the resulting hydrophilic gel matrix.

Because the formulations described herein do not dose dump in the presence of ethanol, they can be used for formulation of drugs that are at risk to be taken with ethanol, such as abuse-potential drugs and drugs prescribed to alcohol and/or drug abusers, or drugs that produce harmful or lethal side effects if over-dosed. Examples of such drugs include opioids.

In addition, patients being treated for conditions such as panic disorder (with or without agoraphobia), bipolar disorder (manic depressive illness), acute manic or mixed episodes associated with bipolar disorder, epilepsy, migraine, attention deficit hyperactivity disorder (ADHD), depression and/or pain may be more likely to consume alcohol compared to the general population. This could be a result of the patients' desire to experience the euphoric effects from inebriation and/or to eliminate or alleviate the symptoms of their condition, such as pain.

Due to the slow release of the drug from the formulations described herein, the patient (e.g., a drug addict) would not experience the euphoria that would be immediately available by abusing conventional formulations (e.g., opioid formulations) by oral inhalation/ingestion or oral ingestion with an alcoholic beverage. Accordingly, the drug formulations described herein would not be abused by patients or their potential for abuse would be significantly reduced (e.g., when compared to conventional opioid formulations).

Additionally, because the drug is released slowly from a sustained release formulation over an extended period of time, many sustained release formulations contain relatively high amounts of the drug. Sustained release formulations containing high amounts of drugs can be more harmful to a patient when they fail compared to immediate release formulations, which generally contain smaller amounts of the drug. Therefore, the drug formulations described herein can increase safety of drugs that can be harmful and/or lethal at higher than therapeutically beneficial levels.

Sustained Release Delivery System

The sustained release delivery system comprises at least one hydrophilic compound. The hydrophilic compound may form a gel matrix that releases the drug at a sustained rate upon exposure to liquids.

The hydrophilic compound can be any hydrophilic compound that in combination with a cross-linking agent produces an ethanol-insoluble or substantially ethanol-insoluble matrix. Exemplary hydrophilic compounds include gums, cellulose ethers, protein-derived compounds, and mixtures thereof. Exemplary gums include heteropolysaccharide gums and homopolysaccharide gums, such as xanthan, tragacanth, pectins, acacia, karaya, alginates, agar, guar, hydroxypropyl guar, carrageenan, locust bean gums, and gellan gums. Exemplary cellulose ethers include hydroxyalkyl celluloses, carboxyalkyl celluloses, hydroxypropyl celluloses, hydroxypropylmethyl-celluloses, carboxy methylcelluloses, and mixtures thereof.

In some embodiments, the hydrophilic compound is a gum, a heteropolysaccharide gum, a xanthan gum, or a derivative thereof. Derivatives of xanthan gum include, for example, deacylated xanthan gum, the carboxymethyl esters of xanthan gum, and the propylene glycol esters of xanthan gum.

In another embodiment, the sustained release delivery system further comprises at least one cross-linking agent. The cross-linking agent is a compound that is capable of cross-linking the hydrophilic compound to form an ethanol-insoluble or substantially ethanol-insoluble gel matrix in the presence of liquids. In various embodiments, the sustained release delivery system generally comprises the cross-linking agent in an amount of about 0.5% to about 80% by weight, in an amount of about 2% to about 54% by weight, in an amount of about 20% to about 30% by weight more, or in an amount of about 25% by weight.

Exemplary cross-linking agents include homopolysaccharides. Exemplary homopolysaccharides include galactomannan gums, such as guar gum, hydroxypropyl guar gum, and locust bean gum. In some embodiments, the cross-linking agent is a locust bean gum, a guar gum, or a mixture thereof. In other embodiments, the cross-linking agents may be alginic acid derivatives or hydrocolloids.

When the sustained release delivery system comprises at least one hydrophilic compound and at least one cross-linking agent, the ratio of the hydrophilic compound to the cross-linking agent may be from about 1:9 to about 9:1, or from about 1:3 to about 3:1.

The sustained release delivery system described herein may comprise one or more cationic cross-linking compounds. The cationic cross-linking compound may be used instead of or in addition to the cross-linking agent. The cationic cross-linking compounds may be used in an amount sufficient to cross-link the hydrophilic compound to form an ethanol-insoluble or substantially ethanol-insoluble gel matrix in the presence of liquids. In some embodiments, the cationic cross-linking compound is present in the sustained release delivery system in an amount of about 0.5% to about 30% by weight, or from about 5% to about 20% by weight.

Exemplary cationic cross-linking compounds include monovalent metal cations, multivalent metal cations, and inorganic salts, including alkali metal and/or alkaline earth metal sulfates, chlorides, borates, bromides, citrates, acetates, lactates, and mixtures thereof. For example, the cationic cross-linking compound may be one or more of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, or mixtures thereof.

When the sustained release delivery system comprises at least one hydrophilic compound and at least one cationic cross-linking compound, the ratio of the hydrophilic compound to the cationic cross-linking compound may be from about 1:9 to about 9:1, or from about 1:3 to about 3:1.

Two properties of compounds (e.g., the at least one hydrophilic compound and the at least one cross-linking agent, or the at least one hydrophilic compound and at least one cationic cross-linking compound) that form a gel matrix upon exposure to liquids are fast hydration of the compounds/agents and a gel matrix having a high gel strength. These two properties, which are useful to achieve a slow release gel matrix, are provided by the particular combination of compounds (e.g., the at least one hydrophilic compound and the at least one cross-linking agent, or the at least one hydrophilic compound and the at least one cationic cross-linking compound). For example, hydrophilic compounds (e.g., xanthan gum) have excellent water-wicking properties that provide fast hydration. The combination of hydrophilic compounds with materials that are capable of cross-linking the rigid helical ordered structure of the hydrophilic compound (e.g., cross-linking agents and/or cationic cross-linking compounds) thereby act synergistically to provide a higher than expected viscosity (i.e., high gel strength) of the gel matrix. The fast hydration and high gel strength of the matrix are believed to contribute to the ethanol-resistant properties of the matrix.

The sustained release delivery system may further comprise one or more pharmaceutical diluents known in the art. Exemplary pharmaceutical diluents include monosaccharides, disaccharides, polyhydric alcohols and mixtures thereof. Pharmaceutical diluents also include, for example, starch, lactose, dextrose, sucrose, microcrystalline cellulose, sorbitol, xylitol, fructose, and mixtures thereof. In other embodiments, the pharmaceutical diluent is water-soluble, such as lactose, dextrose, sucrose, or mixtures thereof. The ratio of pharmaceutical diluent to hydrophilic compound is generally from about 1:8 to about 8:1, or from about 1:3 to about 3:1. The sustained release delivery system generally comprises one or more pharmaceutical diluents in an amount of about 20% to about 80% by weight, or about 35% by weight. In other embodiments, the sustained release delivery system comprises one or more pharmaceutical diluents in an amount of about 40% to about 80% by weight.

The sustained release delivery system described herein may further comprise one or more hydrophobic polymers. The hydrophobic polymers may be used in an amount sufficient to slow the hydration of the hydrophilic compound without disrupting it and without disturbing the hydrophilic character of the matrix. For example, the hydrophobic polymer may be present in the sustained release delivery system in an amount of about 0.5% to about 20% by weight, in an amount of about 2% to about 10% by weight, in an amount of about 3% to about 7% by weight, or in an amount of about 5% by weight, as long as the presence of the hydrophobic polymer in the tablet does not adversely affect the release of the drug from the matrix in the presence of alcohol.

Exemplary hydrophobic polymers include alkyl celluloses (e.g., C₁₋₆ alkyl celluloses, carboxymethylcellulose), and other hydrophobic cellulosic materials. The hydrophobic polymer may be, for example, methyl cellulose, ethyl cellulose, or propyl cellulose.

The compositions described herein may be further admixed with one or more lubricants (such as magnesium stearate), one or more buffering agents, one or more colorants, and/or other conventional ingredients.

In some embodiments, the sustained release formulations comprising at least one opioid are orally administrable solid dosage formulations, for example, tablets, capsules comprising a plurality of granules, sublingual tablets, powders, or granules. The tablets may have an enteric coating or a hydrophilic coating.

The rate of release of the drug from the gel matrix depends on the drug's partition coefficient between the components of the gel matrix and the aqueous phase within the gastrointestinal tract. In the compositions described herein, the weight ratio of drug to hydrophilic compound is generally in the range of about 1:0.5 to about 1:25, or in the range of about 1:0.5 to about 1:20. The sustained release delivery system generally comprises the hydrophilic compound in an amount of about 20% to about 80% by weight, in an amount of about 20% to about 60% by weight, in an amount of about 40% to about 60% by weight, or in an amount of about 50% by weight.

The sustained release delivery system in the compositions described herein may be prepared by dry granulation or wet granulation, before the drug is added, although the components may be held together by an agglomeration technique to produce an acceptable product. In the wet granulation technique, the components (e.g., hydrophilic compounds, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules of the sustained release delivery system. Thereafter, the sustained release delivery system is mixed in the desired amounts with a drug, for example an opioid and, optionally, one or more wetting agents, one or more lubricants, one or more buffering agents, one or more coloring agents, or other conventional ingredients, to produce a granulated composition. The sustained release delivery system and the drug may be blended with, for example, a high shear mixer. In some embodiments, the drug is finely and homogeneously dispersed in the sustained release delivery system. The granulated composition, in an amount sufficient to make a uniform batch of tablets, is subjected to tableting in a conventional production scale tableting machine at normal compression pressures, i.e., about 2,000-16,000 psi. The mixture should not be compressed to a point where there is subsequent difficulty with hydration upon exposure to liquids. Methods for preparing sustained release delivery systems are described in U.S. Pat. Nos. 4,994,276, 5,128,143, 5,135,757, 5,455,046, 5,512,297 and 5,554,387, the disclosures of which are incorporated by reference herein in their entirety.

Upon preparation, granulated compositions and/or tablets, or a sample thereof, can be tested to verify that the formulation is ethanol-resistant. This could be done, for example, using the in vitro methods described below.

In some embodiments, the average particle size of the granulated composition is from about 50 microns to about 400 microns, or from about 185 microns to about 265 microns. The average density of the granulated composition is from about 0.3 g/ml to about 0.8 g/ml, or from about 0.5 g/ml to about 0.7 g/ml. The tablets formed from the granulations are generally from about 6 to about 8 kg hardness. The average flow of the granulations are from about 25 to about 40 g/sec.

In other embodiments, an inner core comprising at least one drug may be coated with a sustained release coating. For example, the inner core comprising the drug may be coated with a sustained release film, which, upon exposure to liquids, releases the drug from the core at a sustained rate.

In one embodiment, the sustained release coating comprises a small amount of at least one water insoluble compound, in an amount less than 5% by weight. The water insoluble compound may be a hydrophobic polymer. Exemplary hydrophobic polymers include alkyl celluloses (e.g., C₁₋₆ alkyl celluloses, carboxymethylcellulose) and other hydrophobic cellulosic materials or compounds. In some embodiments, the hydrophobic polymer is, methyl cellulose, ethyl cellulose or propyl cellulose. The sustained release formulations described herein may be coated with a water insoluble compound to a weight gain from about 1% to about 5% by weight.

The sustained release coating may also contain at least one water-soluble compound, such as hydroxypropylmethylcelluloses, or mixtures thereof. The sustained release coating may comprise at least one water-soluble compound in an amount from about 1% to about 6% by weight, or in an amount of about 3% by weight.

In other embodiments, the compositions comprising at least one drug and a sustained release delivery system, as described herein, are coated with a sustained release coating, as described herein.

In any of the embodiments described herein, the compositions comprising a drug and a sustained release delivery system, as described herein, may optionally be coated with a hydrophilic coating, which may be applied above or beneath the sustained release film and/or above or beneath the enteric coating. In one embodiment, the hydrophilic coating comprises hydroxypropylmethylcellulose.

Without intending to be bound by any theory of the invention, upon oral ingestion of the drug sustained release formulation and contact of the formulation with gastrointestinal fluids, the sustained release formulation swells and gels to form a hydrophilic gel matrix from which the drug is released. The swelling of the gel matrix causes a reduction in the bulk density of the formulation and provides the buoyancy necessary to allow the gel matrix to float on the stomach contents to provide a slow delivery of the drug. The hydrophilic matrix, the size of which is dependent upon the size of the original formulation, can swell considerably and become obstructed near the opening of the pylorus. Because the drug is dispersed throughout the formulation (and consequently throughout the gel matrix), a constant amount of the drug can be released per unit time in vivo by dispersion or erosion of the outer portions of the hydrophilic gel matrix. This phenomenon is referred to as a zero order release profile or zero order kinetics. The process continues, with the gel matrix remaining buoyant in the stomach, until substantially all of the drug is released.

Without intending to be bound by any theory of the invention, the chemistry of certain of the components of the formulation, such as the hydrophilic compound (e.g., xanthan gum), is such that the components are considered to be self-buffering agents which are substantially insensitive to the solubility of the drugs and the pH changes along the length of the gastrointestinal tract. Moreover, the chemistry of the components is believed to be similar to certain known muco-adhesive substances, such as polycarbophil. Muco-adhesive properties are desirable for buccal delivery systems. Thus, it may be possible that the sustained release formulation could potentially loosely interact with the mucin in the gastrointestinal tract and thereby provide another mode by which a constant rate of delivery of the drug is achieved.

The two phenomena discussed above (hydrophilic gel matrix and muco-adhesive properties) are possible mechanisms by which the sustained release formulations described herein could interact with the mucin and fluids of the gastrointestinal tract and provide a constant rate of delivery of the drugs. These properties appear to be substantially unmodified in the presence of ethanol.

While the compositions described herein may be provided, administered, or prescribed as the sole active pharmaceutical composition in the methods described herein, they can also be used in combination with one or more compounds/compositions that are known to be therapeutically effective against a condition, or any other disease or symptom.

### EXAMPLES

The following examples are for purposes of illustration only and are not intended to limit the scope of the appended claims.

The experiments were performed with Albuterol Sulfate, which has dosage, solubility and other physicochemical properties similar to opioids, such as oxymorphone and oxycodone.

### Example 1

### Preparation of the Formulation

A sustained release formulation was prepared by first screening Albuterol Sulfate, and Prosolv® 90M (Microcrystalline Cellulose, JRS Pharma LP, Patterson, N.Y.) separately through a #30 Mesh sieve. Albuterol Sulfate and TIMERx-N® (Xantham Gum and Locust Bean Gum, Penwest Pharmaceuticals Co., Patterson, N.Y.) were blended for ten minutes in a Patterson-Kelley P/K Blendmaster V-Blender. Prosolv® 90M (augmented Microcrystalline Cellulose, JRS Pharma LP, Patterson, N.Y.) and Pruv™ (Sodium Stearyl Fumarate, NF, JRS Pharma LP, Patterson, N.Y.) were added to this mixture successively, blending for five minutes between each addition. The blended granulation was compressed to 224.0 mg and 11 Kp hardness on a tablet press using a Stokes RB-2 5/16″ round standard concave beveled edge. The final tablet composition is listed in the table below:

| Component | % | mg/tablet |
|---|---|---|
| Albuterol Sulfate | 17.9 | 40.0 |
| TIMERx-N ® | 71.4 | 160.0 |
| Prosolv ® 90M | 8.9 | 20.0 |
| Pruv ™ | 1.8 | 4.0 |

### Example 2

### Dissolution Properties of Different Lots of TIMERx-N®

To assess the variability in dissolution profiles among different grades of TIMERx-N®, the following experiment was conducted.

Tablets of TIMERx-N® formulations of Example 1, were prepared as described in Example 1 using three different lots of TIMERx-N®. Dissolution profiles of each formulation were evaluated using a USP Type II dissolution apparatus in 900 mL of 50 mM potassium phosphate buffer (pH 4.5). The solution was stirred at 50 r.p.m. A series of samples of about 1.5 mL were withdrawn at predetermined intervals for a period of up to 14 hours.

Drug release for all formulations was monitored by RP-HPLC using a Waters Symmetry® C18 column (4.6×250 mm) (or equivalent) preceded by a Phenomenex® SecurityGuard™ C18 (4×3.0 mm) guard column. Monitoring wavelength was set to 226 nm. The mobile phase consisted of buffer:acetonitrile:methanol in 85:10:5 v/v ratios. The buffer consisted of 1 mL triethylamine and 1 mL trifluoroacetic acid in 1 L of H₂O. The column temperature was either ambient temperature or 30° C. and the flow rate was set to 1.5 mL/min. To determine the percentage of drug released at each timepoint, the concentration of the sample taken at that timepoint was compared to the concentration of a standard solution. The standard solution was prepared by dissolving 45 mg of albuterol sulfate in 100 mL of 50 mM potassium phosphate buffer (pH 4.5) and then taking 5 mL of this solution and diluting it to 50 mL with more of 50 mM potassium phosphate buffer (pH 4.5).

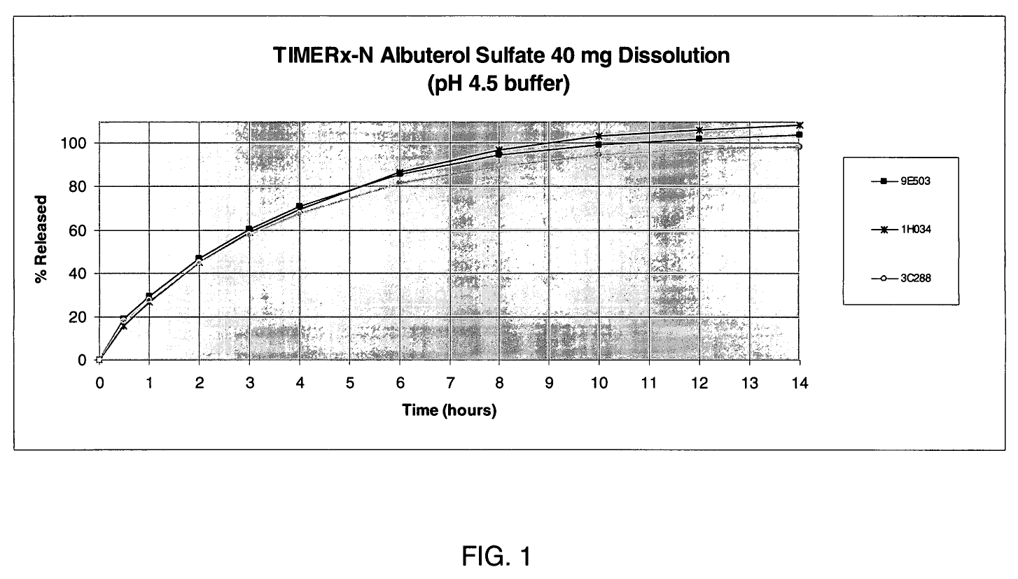

FIG. 1 is a graphical depiction of the dissolution profiles of Formulation 1, Formulation 2, and Formulation 3. Each data point is a result of 3-6 runs.

As can be seen from FIG. 1, dissolution profiles exhibit small variations between different lots of TIMERx-N®, but all lots show a similar sustained release dissolution profile in phosphate buffer (pH 4.5). The entire amount of drug is released approximately 10-14 hours after beginning of dissolution. These formulations, therefore, exhibit expected dissolution profiles for sustained release formulations of this type.

### Example 3

### Dissolution Properties in the Presence of Ethanol

To test the formulations described herein for resistance to ethanol, dissolution profiles of formulations prepared according to Example 1 and assayed according to Example 2, were also recorded in the presence of ethanol. A medium of 40% ethanol and 60% 0.1 M HCl was used as a model of dissolution in the presence of alcohol. 0.1M HCl was chosen to mimic the biological environment of upper GI tract/stomach area, where the sustained release formulation first begins to release the drug.

Dissolution experiments were performed using a USP II Type dissolution apparatus according to methods described above. The results of the dissolution experiments are shown in Table 1.

TABLE 1
Percentage of albuterol sulfate released as a function of time

| | | Phosphate | 40% Ethanol, | Phosphate | 40% Ethanol, | Phosphate | 40% Ethanol, |
|---|---|---|---|---|---|---|---|
| Dissolution | | buffer | 60% 0.1M HCl | buffer | 60% 0.1M HCl | buffer | 60% 0.1M HCl |
| Medium | | Formulation | Formulation | Formulation | Formulation | Formulation | Formulation |
| Time | | 1 | 1 | 2 | 2 | 3 | 3 |
| 0.5 | hr | 18.7 | 16.1 | 15.7 | 28.8 | 17.8 | 15.8 |
| 1 | hr | 29.6 | 25.5 | 26.8 | 38.1 | 27.5 | 24.1 |
| 2 | hrs | 46.9 | 40.3 | 45.2 | 51.5 | 45.1 | 34.9 |
| 3 | hrs | 60.2 | 53.0 | 58.7 | 63.6 | 57.9 | 44.6 |
| 4 | hrs | 70.9 | 63.7 | 69.6 | 76.9 | 67.7 | 52.5 |
| 6 | hrs | 85.4 | 78.0 | 86.5 | 92.8 | 81.5 | 66.0 |
| 8 | hrs | 94.2 | 87.6 | 96.8 | 99.0 | 89.4 | 74.2 |
| 10 | hrs | 98.9 | 96.6 | 103.3 | 101.7 | 94.3 | 80.9 |
| 12 | hrs | 101.7 | 103.1 | 105.9 | 103.5 | 96.9 | 85.5 |
| 14 | hrs | 103.7 | 106.5 | 108.0 | 105.0 | 98.1 | 88.9 |

| | | | | | | |
|---|---|---|---|---|---|---|
| Assay | 104.4 | 111.5 | 108.6 | 108.4 | 94.7 | 97.3 |

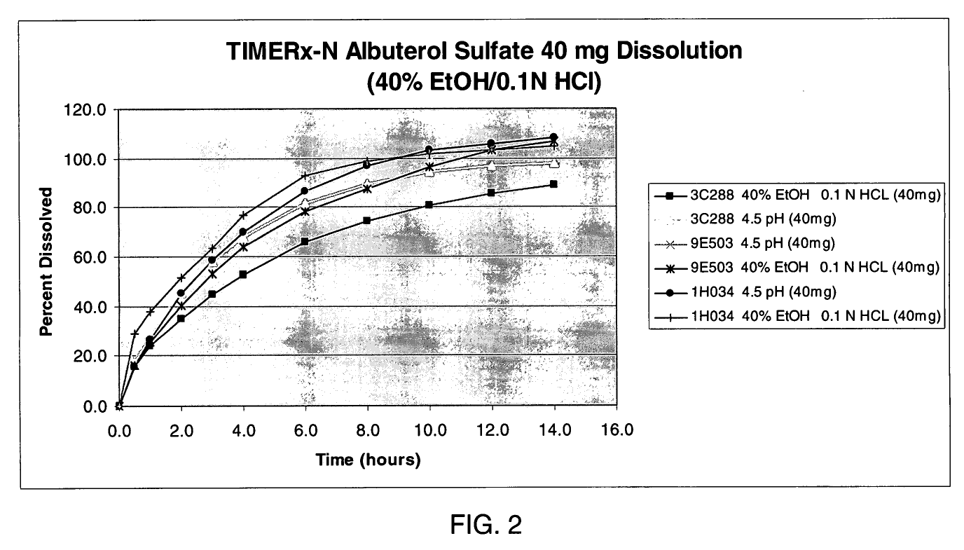

FIG. 2 is a graphical depiction of the dissolution profiles of Formulation 1, Formulation 2, and Formulation 3 in the presence (40% ethanol, 60% 0.1M HCl v/v) and absence (50 mM potassium phosphate buffer, pH 4.5) of ethanol. As can be seen from FIG. 2 and Table 1, all formulations essentially retained their sustained release dissolution profiles when the potassium phosphate buffer (pH 4.5) was substituted for a 40% ethanol/60% 0.1 M HCl solution. While some lots of TIMERx-N® (e.g., Formulation 3) exhibited greater sensitivity to ethanol than others (Formulation 1 and Formulation 2), the differences were small and the final assay values fell within the quality control specifications for tablet content uniformity standards of 85-115% (±6%) set forth by the United States Pharmacopeia (USP). (Uniformity of dosage units, United States Pharmacopeia 24/National Formulary 19, Rockville, Md.: United States Pharmacopeial Convention, Inc.; 1999:2000-3)

In all cases, the formulations retained their sustained release characteristics in the presence of 40% ethanol.

Similar results are expected to be obtained with other chemical entities, because the properties of the sustained release system affect the dissolution properties of the formulation to a significantly larger extent than the nature of the drug in the formulation. Ethanol dissolution testing is contemplated to become a standard procedure in development of new sustained release products.

The patents, patent applications, and publications cited herein are incorporated by reference herein in their entirety. Various modifications of the invention, in addition to those described herein, will be apparent to one skilled in the art from the foregoing description. Such modifications are intended to fall within the scope of the appended claims.

What is claimed is:

1. A method of preventing dose-dumping of a drug in the presence of ethanol comprising: providing a patient likely to consume ethanol while being treated with the drug an effective amount of the drug in the form of an ethanol-resistant sustained release formulation comprising:
the drug; and
a sustained release delivery system,
the delivery system comprising at least one heteropolysaccharide gum, at least one homopolysaccharide gum and at least one pharmaceutical diluent, wherein the ethanol-resistant sustained release formulation essentially retains its sustained release dissolution profile in the presence of ethanol.

2. The method of claim 1, wherein the patient has a history of substance abuse.

3. The method of claim 2, wherein the substance abuse is alcohol abuse.

4. The method of claim 2, wherein the substance abuse is drug abuse.

5. The method of claim 1, wherein the ethanol-resistant sustained release formulation is a solid dosage formulation.

6. The method of claim 5, wherein the solid dosage formulation retains its dissolution profile when crushed or powdered.

7. The method of claim 5, wherein the solid dosage formulation forms a gel matrix with muco-adhesive properties when crushed or powdered upon contact with a fluid.

8. The method of claim 5, wherein the solid dosage formulation forms a viscous solution when crushed or powdered upon contact with a fluid.

9. The method of claim 5, wherein the solid dosage formulation is a tablet.

10. The method of claim 1, wherein the sustained release delivery system further comprises at least one hydrophobic polymer in an amount of less than about 5% by weight.

11. The method of claim 1, wherein the delivery system further comprises at least one cationic cross-linking compound selected from monovalent cations, multivalent cations, and salts.

12. The method of claim 1, wherein the drug is an anti-depressant.

13. The method of claim 1, wherein the drug is a drug used to treat bipolar disorder, panic disorder, epilepsy, migraine, and/or attention deficit hyperactivity disorder.

14. The method of claim 1, wherein the drug is an opioid.

15. The method of claim 14, wherein the opioid is a mu-agonist or a mixed mu-agonist/antagonist.

16. The method of claim 14, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

17. A method of preventing dose-dumping of a drug in the presence of ethanol comprising: providing a patient likely to consume ethanol while being treated with the drug an effective amount of the drug in the form of an ethanol-resistant sustained release formulation comprising:
the drug; and
a sustained release delivery system,
the delivery system comprising at least one heteropolysaccharide gum, at least one cationic cross-linking compound selected from monovalent metal cations, multivalent metal cations and salts, and at least one pharmaceutical diluent, wherein the ethanol-resistant sustained release formulation essentially retains its sustained release dissolution profile in the presence of ethanol.

18. The method of claim 17, wherein the patient has a history of substance abuse.

19. The method of claim 18, wherein substance abuse is alcohol abuse.

20. The method of claim 18, wherein the substance abuse is drug abuse.

21. The method of claim 17, wherein the ethanol-resistant sustained release formulation is a solid dosage formulation.

22. The method of claim 21, wherein the solid dosage formulation retains its dissolution profile when crushed or powdered.

23. The method of claim 21, wherein the solid dosage formulation forms a gel matrix with muco-adhesive properties when crushed or powdered upon contact with a fluid.

24. The method of claim 21, wherein the solid dosage formulation forms a viscous solution when crushed or powdered upon contact with a fluid.

25. The method of claim 21, wherein the solid dosage formulation is a tablet.

26. The method of claim 17, wherein the sustained release delivery system further comprises at least one hydrophobic polymer in an amount of less than about 5% by weight.

27. The method of claim 17, wherein the at least one cationic cross-linking compound is a sodium salt.

28. The method of claim 17, wherein the drug is an anti-depressant.

29. The method of claim 17, wherein the drug is a drug used to treat bipolar disorder, panic disorder, epilepsy, migraine, and/or attention deficit hyperactivity disorder.

30. The method of claim 17, wherein the drug is an opioid.

31. The method of claim 30, wherein the opioid is a mu-agonist or a mixed mu-agonist/antagonist.

32. The method of claim 30, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

33. A method of improving safety of a drug compared to conventional sustained release formulations in the presence of ethanol comprising providing a patient likely to consume ethanol while being treated with the drug an effective amount of the drug in the form of an improved-safety ethanol-resistant sustained release formulation comprising:
the drug; and
a sustained release delivery system,
the sustained release delivery system comprising at least one heteropolysaccharide gum, at least one homopolysaccharide gum and at least one pharmaceutical diluent, wherein the improved-safety is a result of ethanol-resistant sustained release properties of the formulation.

34. The method of claim 33, wherein the patient has a history of substance abuse.

35. The method of claim 34, wherein the substance abuse is alcohol abuse.

36. The method of claim 34, wherein the substance abuse is drug abuse.

37. The method of claim 33, wherein the ethanol-resistant sustained release formulation is a solid dosage formulation.

38. The method of claim 37, wherein the solid dosage formulation retains its dissolution profile when crushed or powdered.

39. The method of claim 37, wherein the solid dosage formulation forms a gel matrix with muco-adhesive properties when crushed or powdered upon contact with a fluid.

40. The method of claim 37, wherein the solid dosage formulation forms a viscous solution when crushed or powdered upon contact with a fluid.

41. The method of claim 37, wherein the solid dosage formulation is a tablet.

42. The method of claim 33, wherein the sustained release delivery system further comprises at least one hydrophobic polymer in an amount of less than about 5% by weight.

43. The method of claim 33, wherein the delivery system further comprises at least one cationic cross-linking compound selected from monovalent cations, multivalent cations, and salts.

44. The method of claim 33, wherein the drug is an anti-depressant.

45. The method of claim 33, wherein the drug is a drug used to treat bipolar disorder, panic disorder, epilepsy, migraine, and/or attention deficit hyperactivity disorder.

46. The method of claim 33, wherein the drug is an opioid.

47. The method of claim 46, wherein the opioid is a mu-agonist or a mixed mu-agonist/antagonist.

48. The method of claim 46, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

49. A method of improving safety of a drug compared to conventional sustained release formulations in the presence of ethanol comprising providing a patient likely to consume ethanol while being treated with the drug an effective amount of the drug in the form of an improved-safety ethanol-resistant sustained release formulation comprising:
the drug; and
a sustained release delivery system,
the delivery system comprising at least one heteropolysaccharide gum, at least one cationic cross-linking compound selected from monovalent metal cations, multivalent metal cations and salts, and at least one pharmaceutical diluent, wherein the improved-safety is a result of ethanol-resistant sustained release properties of the formulation.

50. The method of claim 49, wherein the patient has a history of substance abuse.

51. The method of claim 50, wherein substance abuse is alcohol abuse.

52. The method of claim 50, wherein the substance abuse is drug abuse.

53. The method of claim 49, wherein the ethanol-resistant sustained release formulation is a solid dosage formulation.

54. The method of claim 53, wherein the solid dosage formulation retains its dissolution profile when crushed or powdered.

55. The method of claim 53, wherein the solid dosage formulation forms a gel matrix with muco-adhesive properties when crushed or powdered upon contact with a fluid.

56. The method of claim 53, wherein the solid dosage formulation forms a viscous solution when crushed or powdered upon contact with a fluid.

57. The method of claim 53, wherein the solid dosage formulation is a tablet.

58. The method of claim 49, wherein the sustained release delivery system further comprises at least one hydrophobic polymer in an amount of less than about 5% by weight.

59. The method of claim 49, wherein the at least one cationic cross-linking compound is a sodium salt.

60. The method of claim 49, wherein the drug is an anti-depressant.

61. The method of claim 49, wherein the drug is a drug used to treat bipolar disorder, panic disorder, epilepsy, migraine, and/or attention deficit hyperactivity disorder.

62. The method of claim 49, wherein the drug is an opioid.

63. The method of claim 62, wherein the opioid is a mu-agonist or a mixed mu-agonist/antagonist.

64. The method of claim 62, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

65. A method for making a solid dosage ethanol-resistant sustained release formulation comprising:
at least one drug; and
a sustained release delivery system,
wherein the sustained release delivery system comprises at least one heteropolysaccharide gum, at least one homopolysaccharide gum, and at least one pharmaceutical diluent, the method comprising:
mixing the at least one heteropolysaccharide gum, the at least one homopolysaccharide gum and the at least one pharmaceutical diluent to form granules;
mixing the granules with at least one drug or a pharmaceutically acceptable salt thereof to form a granulated composition;
applying pressure to the granulated composition to make the formulation; and
recording a dissolution profile of the ethanol-resistant formulation in an ethanol-containing solution.

66. The method of claim 65, further comprising applying an outer coating onto at least part of the sustained release formulation.

67. The method of claim 65, wherein the at least one drug is an opioid.

68. The method of claim 67, wherein the opioid is a mu-agonist or a mixed mu-agonist/antagonist.

69. The method of claim 67, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

70. A method for making a solid dosage ethanol-resistant formulation comprising:
at least one drug; and
a sustained release delivery system,
wherein the sustained release delivery system comprises at least one heteropolysaccharide gum, at least one cationic cross-linking compound selected from monovalent metal cations, multivalent metal cations and salts, and at least one pharmaceutical diluent, the method comprising:
mixing the at least one heteropolysaccharide gum, the at least one cationic cross-linking compound selected from monovalent metal cations, multivalent metal cations and salts, and the at least one pharmaceutical diluent to form granules;
mixing the granules with at least one drug or a pharmaceutically acceptable salt thereof to form a granulated composition;
applying pressure to the granulated composition to make the formulation; and
recording a dissolution profile of the ethanol-resistant formulation in an ethanol-containing solution.

71. The method of claim 70, further comprising applying an outer coating onto at least part of the sustained release formulation.

72. The method of claim 70, wherein the at least one drug is an opioid.

73. The method of claim 72, wherein the opioid is a mu-agonist or a mixed mu-agonist/antagonist.

74. The method of claim 72, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

75. A method for treating a patient comprising providing a patient having a history of substance abuse an effective amount of the drug in the form of an ethanol-resistant sustained release formulation comprising:
at least one drug; and
a sustained release delivery system,
the delivery system comprising at least one heteropolysaccharide gum, at least one homopolysaccharide gum, and at least one pharmaceutical diluent, wherein the ethanol-resistant sustained release formulation essentially retains its sustained release dissolution profile in the presence of ethanol.

76. The method of claim 75, wherein the substance abuse is alcohol abuse.

77. The method of claim 75, wherein the substance abuse is drug abuse

78. The method of claim 75, wherein the ethanol-resistant formulation is a solid dosage formulation.

79. The method of claim 78, wherein the solid dosage formulation retains its dissolution profile when crushed or powdered.

80. The method of claim 78, wherein the solid dosage formulation forms a gel matrix with muco-adhesive properties when crushed or powdered upon contact with a fluid.

81. The method of claim 78, wherein the solid dosage formulation forms a viscous solution when crushed or powdered upon contact with a fluid.

82. The method of claim 78, wherein the solid dosage formulation is a tablet.

83. The method of claim 75, wherein the delivery system further comprises at least one hydrophobic polymer in an amount of less than about 5% by weight.

84. The method of claim 75, wherein the delivery system further comprises at least one cationic cross-linking compound selected from monovalent cations, multivalent cations, and salts.

85. The method of claim 75, wherein the drug is an anti-depressant.

86. The method of claim 75, wherein the drug is a drug used to treat bipolar disorder, panic disorder, epilepsy, migraine, and/or attention deficit hyperactivity disorder.

87. The method of claim 75, wherein the drug is an opioid.

88. The method of claim 87, wherein the opioid is a mu-agonist or a mixed mu-agonist/antagonist.

89. The method of claim 87, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

90. A method for treating a patient comprising providing a patient having a history of substance abuse an effective amount of the drug in the form of an ethanol-resistant sustained release formulation comprising:
at least one drug; and
a sustained release delivery system,
the delivery system comprising at least one heteropolysaccharide gum, at least one cationic cross-linking compound selected from monovalent metal cations, multivalent metal cations and salts, and at least one pharmaceutical diluent, wherein the ethanol-resistant sustained release formulation essentially retains its sustained release dissolution profile in the presence of ethanol.

91. The method of claim 90, wherein the substance abuse is alcohol abuse.

92. The method of claim 90, wherein the substance abuse is drug abuse

93. The method of claim 90, wherein the ethanol-resistant formulation is a solid dosage formulation.

94. The method of claim 93, wherein the solid dosage formulation retains its dissolution profile when crushed or powdered.

95. The method of claim 93, wherein the solid dosage formulation forms a gel matrix with muco-adhesive properties when crushed or powdered upon contact with a fluid.

96. The method of claim 93, wherein the solid dosage formulation forms a viscous solution when crushed or powdered upon contact with a fluid.

97. The method of claim 93, wherein the solid dosage formulation is a tablet.

98. The method of claim 90, wherein the delivery system further comprises at least one hydrophobic polymer in an amount of less than about 5% by weight.

99. The method of claim 90, wherein the delivery system further comprises at least one cationic cross-linking compound selected from monovalent cations, multivalent cations, and salts.

100. The method of claim 90, wherein the drug is an anti-depressant.

101. The method of claim 90, wherein the drug is a drug used to treat bipolar disorder, panic disorder, epilepsy, migraine, and/or attention deficit hyperactivity disorder.

102. The method of claim 90, wherein the drug is an opioid.

103. The method of claim 102, wherein the opioid is a mu-agonist or a mixed mu-agonist/antagonist.

104. The method of claim 102, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.
